(19) United States
(12) Patent Application Publication
(10) Pub. No.: US 2023/0321372 A1
(43) Pub. Date: Oct. 12, 2023

(54) DEVICE FOR DISPENSING A FLUID PRODUCT

(71) Applicant: APTAR FRANCE SAS, LE NEUBOURG (FR)

(72) Inventor: David FABIEN, SAINT RENAN (FR)

(73) Assignee: APTAR FRANCE SAS, LE NEUBOURG (FR)

(21) Appl. No.: 18/023,975

(22) PCT Filed: Sep. 1, 2021
(86) PCT No.: PCT/FR2021/051502

§ 371 Date: Feb. 28, 2023

(30) Foreign Application Priority Data
Sep. 1, 2020 (FR) FR2008864

(51) Int. Cl. A61M 15/00

(52) U.S. Cl. CPC A61M 15/0081; A61M 2205/103; A61M 2205/276

(57) ABSTRACT

A device comprising:
a dispenser (1) comprising a reservoir (2) containing the fluid product, a dispensing head (3) provided with a dispensing orifice (4), able to move axially relative to the reservoir (2), and a dispensing member (5) actuated when the reservoir (2) is moved axially upwards relative to said dispensing head (3),
an inner body (10) comprising a hollow cylinder (11) receiving said dispenser (1),
an outer body (20) receiving said inner body (10), a push member (30), and an actuating system;
said push member (30) being movable relative to said inner body (10) between a rest position and an actuating position, a spring (40) being interposed between said inner body (10) and said push member (30), said spring (40) being compressed in the rest position of said push member (30) in order to urge it towards its actuating position,
said actuating system comprising a reset gear (50), a pawl (60), a floating wheel (70), a motor wheel (75), and a motor (80), wherein:
said pawl (60) interacts with said push member (30) to lock it in the rest position, said pawl (60) being moved from its locked position towards a release position by said floating wheel (70) when the motor (80) rotates in a first actuating direction, and
said reset gear (50) interacts with said push member (30), said reset gear (50) being moved by said floating wheel (70) when the motor (80) rotates in a second reset direction in order to return said push member (30) into its rest position.

The present invention relates to a device for dispensing a fluid product.

At present, the administration of potent drugs which are potentially lethal to human beings may be a necessity in certain situations. This is particularly the case for the treatment of particular diseases, or indeed for people in need of palliative treatment in end-of-life contexts. Handling such substances requires great caution and extremely safe administration devices in order to avoid the risk of overdoses which may occur in the event that several consecutive doses are administered close together. Another risk relates to the use of these devices by a person other than the person to whom the treatment is intended, for example children.

Moreover, in particular when the device comprises a manually-actuated pump, the actuation of the device and the quality of the spray obtained are generally dependent on the actuating speed, and therefore highly dependent on the user. Thus, an elderly person, a disabled person or a child is likely to actuate the device with less force and less speed than an able-bodied adult. Moreover, this dependence of the user results in a low reproducibility of the spray from one use to another. This may result in a more or less effective penetration of the drug molecules into the ENT system depending on the user and/or the actuation, with potentially a variable therapeutic effect from one use to another.

Documents JP2011050535, US2002130146, U.S. Pat. No. 3,952,916 and JPS49109924 describe prior-art devices.

The object of this invention is to provide a fluid product dispensing device that does not have the above mentioned disadvantages.

In particular, the object of the present invention is to provide a device for dispensing a fluid product which is safe and which safeguards the user, in particular in order to avoid the risk of overdose.

Another object of the present invention is to provide a fluid product dispensing device that can be used only by authorized persons, in particular by the person having the therapeutic need.

Another object of the present invention is to provide a device for dispensing a fluid product which is locked for a predeterminable period of time between two successive actuations.

Another object of the present invention also is to provide such a device for dispensing a fluid product that guarantees the same spray at each actuation, regardless of the user.

Another object of the present invention is to provide a device for dispensing a fluid product which is robust and reliable in use.

Another object of the present invention is to provide a device for dispensing a fluid product that is simple and inexpensive to manufacture and to assemble.

The present invention therefore provides a device for dispensing a fluid product comprising:
a fluid product dispenser comprising: a reservoir containing a fluid product; a dispensing head that is provided with a dispensing orifice, said dispensing head being able to move axially relative to said reservoir; and a dispensing member, such as a pump or a valve, that is mounted on said reservoir, said dispensing member being actuated when said reservoir is moved axially upwards relative to said dispensing head;
an inner body comprising a hollow cylinder receiving said dispenser,
an outer body receiving said inner body, a push member and an automatic actuating system,
said push member being in contact with said reservoir and mounted axially movable relative to said inner body between a rest position and an actuating position, a spring being interposed between said inner body and said push member, said spring being compressed in the rest position of said push member in order to urge it towards its actuating position;
said automatic actuating system comprising a reset gear, a pawl, a floating wheel, a motor wheel, and a motor, wherein:
said pawl interacts with said push member to lock it in the rest position, said pawl being moved from its locked position towards a release position by said floating wheel when the motor rotates in a first actuating direction, and
said reset gear interacts with said push member, said reset gear being moved by said floating wheel when the motor rotates in a second reset direction in order to return said push member into its rest position.

Advantageously, the rotation of said motor in said first actuating direction moves said floating wheel so as to interact with said pawl, and the rotation of said motor in said second reset direction moves said floating wheel so as to interact with said reset gear.

Advantageously, said reset gear is pivotally mounted on said outer body and comprises a first set of teeth interacting with a set of teeth of said push member, and a second set of teeth interacting with said floating wheel when said motor rotates in said second reset direction.

Advantageously, said pawl is pivotally mounted on said outer body and includes a tooth that interacts with a notch in said push member.

Advantageously, a pawl wheel is mounted on said outer body to rotate about a rotation axis, said pawl comprising an oblong opening mounted about said rotation axis, said pawl wheel interacting with said floating wheel when said motor rotates in said first actuating direction.

Advantageously, said pawl is biased towards its locking position, in particular by a resilient element such as a spring or a resilient blade.

Advantageously, said floating wheel includes a rotation axis that is mounted to move in an oblong opening of said outer body.

Advantageously, said motor wheel is rotatably mounted on said outer body and interacts with said floating wheel when said motor rotates in said first actuating direction or in said second actuating direction.

Advantageously, said motor rotates a worm screw that interacts with said motor wheel.

Advantageously, the device comprises a control module to control said motor.

Advantageously, said control module comprises timing means to prevent, after each actuation of the dispenser, the next actuation of said motor for a predeterminable time.

Advantageously, said timing means locks a control button of the device and/or said motor.

Advantageously, said spring is disposed in said inner body, between a bottom of said inner body and an upper radial flange of said push member.

These characteristics and advantages and others of the present invention appear more clearly from the following detailed description, given by way of non-limiting example, and with reference to the accompanying drawings, and in which:

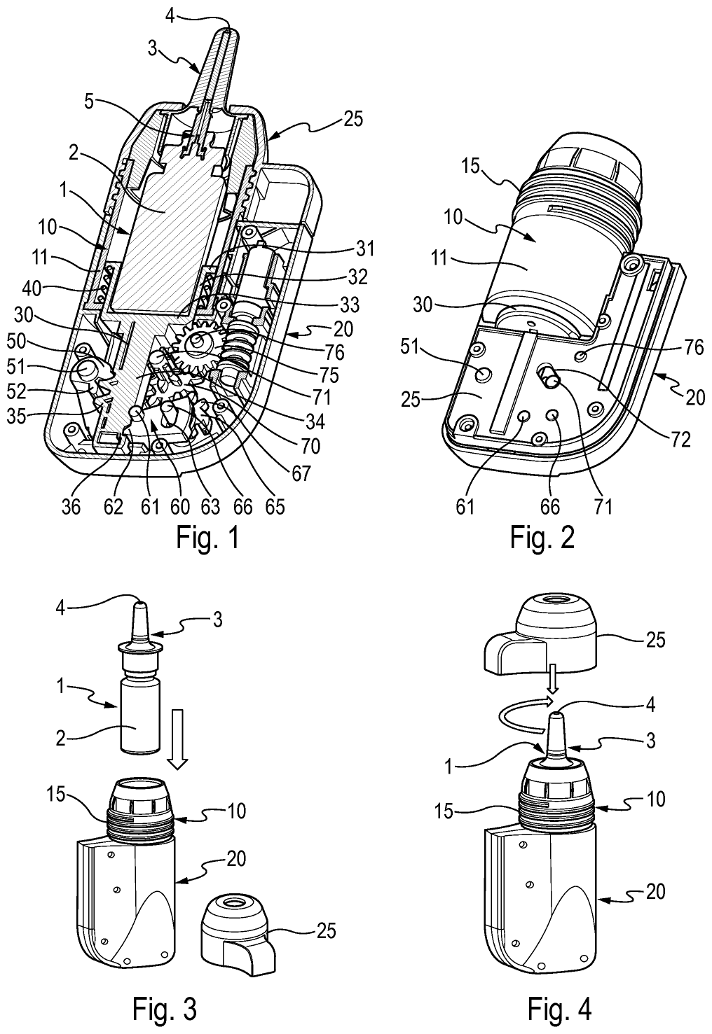

FIG. 1 is a diagrammatic cut perspective view of a dispensing device in accordance with an advantageous embodiment,

FIG. 2 is a diagrammatic fragmentary view of the FIG. 1 device;

FIGS. 3 and 4 are diagrammatic exploded perspective views illustrating the assembly of the fluid product dispenser in the device,

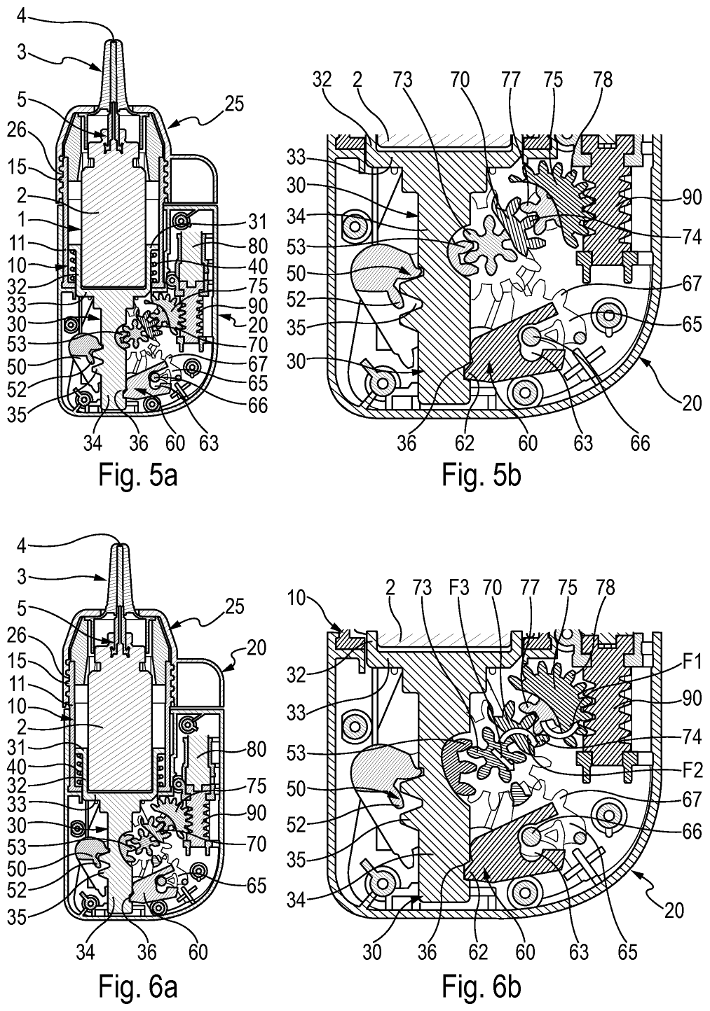

FIG. 5a is a diagrammatic section view of the FIG. 1 device, shown in its rest position;

FIG. 5b is larger-scale diagrammatic view of a detail of the FIG. 5a device actuation mechanism;

FIGS. 6a and 6b are views similar to the views in FIGS. 5a and 5b respectively, at the beginning of actuating stroke;

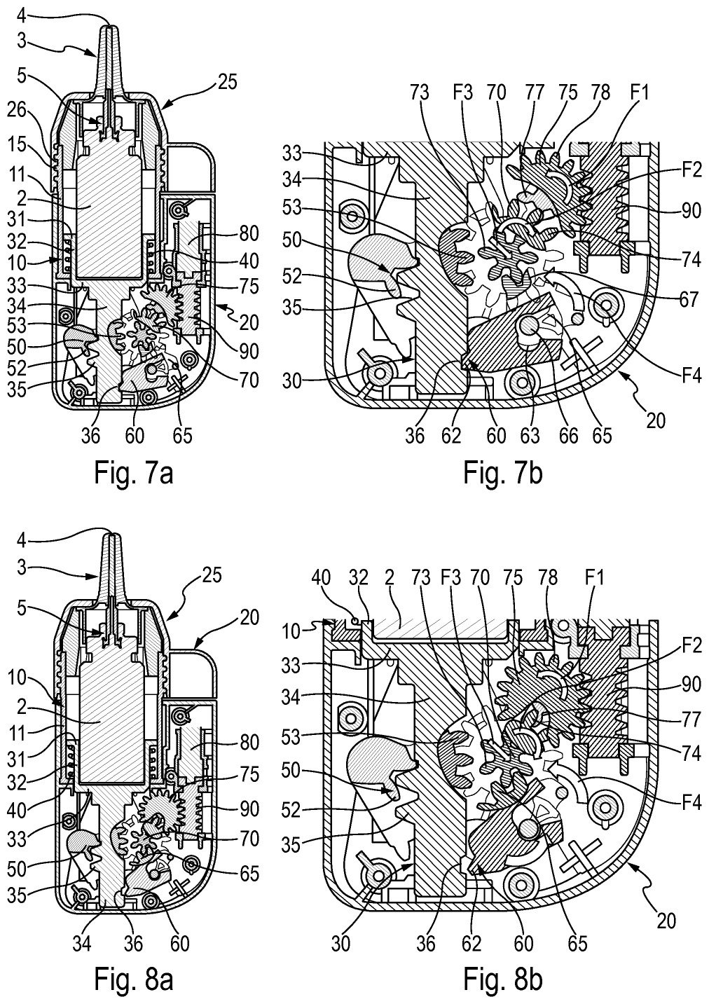

FIGS. 7a and 7b are views similar to the views in FIGS. 6a and 6b respectively, during actuating stroke;

FIGS. 8a and 8b are views similar to the views in FIGS. 7a and 7b respectively, at the end of actuating stroke;

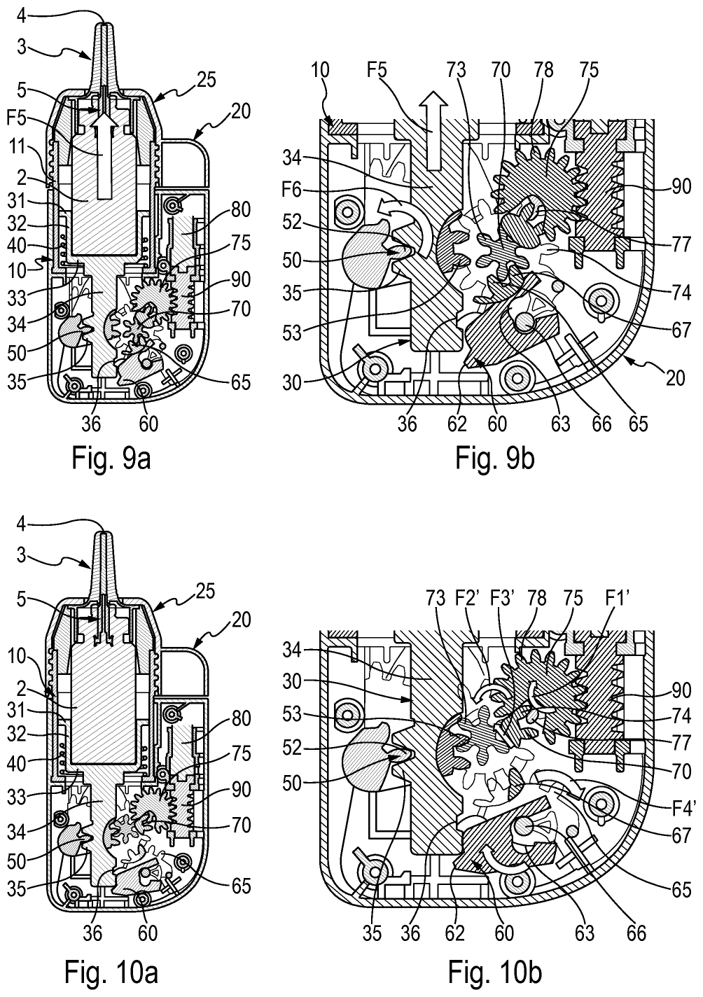

FIGS. 9a and 9b are views similar to the views in FIGS. 8a and 8b, during dispensing of a fluid product dose;

FIGS. 10a and 10b are views similar to the views in FIGS. 9a and 9b, at the start of return stroke;

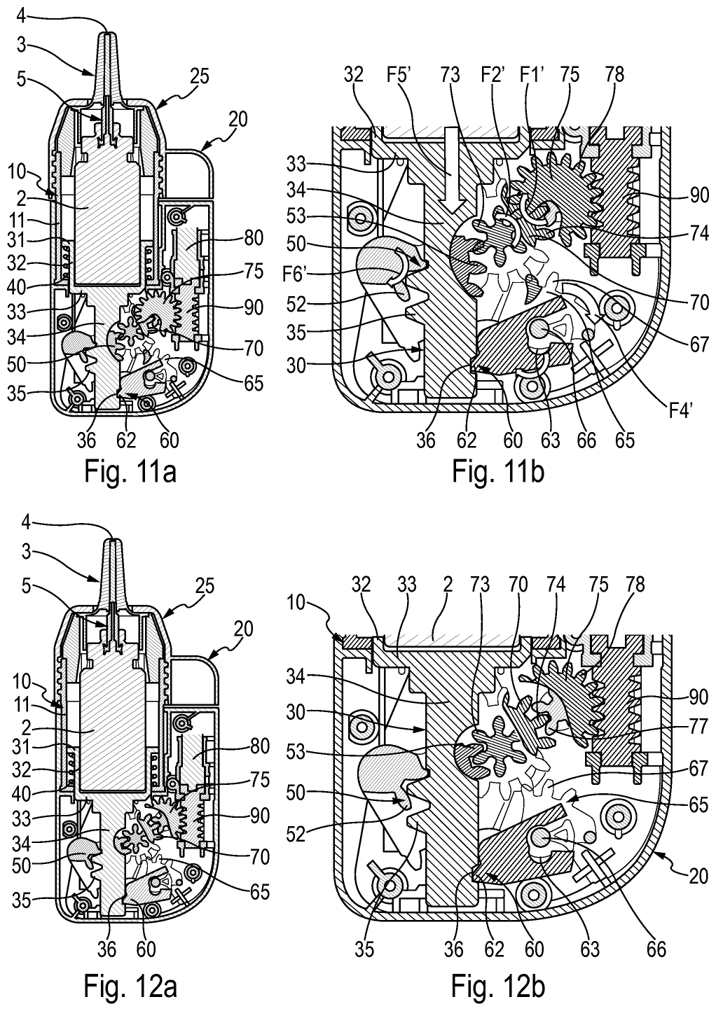

FIGS. 11a to 11b are views similar to the views in FIGS. 10a to 10b, at the end of return stroke;

FIGS. 12a and 12b are views similar to the views in FIGS. 11a and 11b respectively, back in the rest position.

The terms “axial” and “radial” are relative to the longitudinal central axis of the device. The terms “top”, “bottom”, “upper” and “lower” refer to the upright position of the device in particular in FIG. 1.

The principal subject matter of the present invention is a device which authorizes/prohibits the delivery of a dose by locking/releasing the actuation of a fluid product dispenser, while guaranteeing the same spray at each actuation.

The device for dispensing a fluid product shown in the figures comprises a fluid product dispenser 1, advantageously of a standard type. This dispenser 1 comprises a reservoir 2 containing the fluid product and a dispensing head 3 which is axially movable with respect to the reservoir 2. The dispensing head 3 is provided with a dispensing orifice 4. A dispensing member 5, such as a pump or a valve, is mounted on the reservoir 2, said dispensing member being actuated when the reservoir 2 is moved axially upwards relative to the dispensing head 3. Generally, when the reservoir 2 does not contain propellant gas, a dispensing pump is used, and when the reservoir 2 contains propellant gas, a metering valve is used. These two types of dispensing means are well known to the person skilled in the art and since this dispensing means is not directly involved in the present invention, it will not be described in greater detail below. The present invention is, however, particularly adapted to the use of a pump without propellant gas.

The device for dispensing a fluid product also comprises an inner body 10, receiving the dispenser 1, and an outer body 20, fixed to the inner body 10 and containing an electronic control module 90.

The inner body 10 comprises a hollow cylinder 11 which is axially open on either side, with coupling means which are advantageously in the form of a thread 15 on the outer surface of the lower opening.

The outer body 20 is hollow and may have any external shape. The outer body 20 is fixed to the inner body 10 in an appropriate manner, for example by snap fitting. Advantageously, the outer body 20 comprises on the outside one or more displays (not shown), for example a screen, making it possible to display information, such as, for example, instructions for use, battery charging information, etc. The display may also incorporate an actuating zone, such as a touch-sensitive control button, on which the user presses to unlock the device. In a variant, a control button separate from the display could also be provided. Advantageously, the button or the control zone could incorporate fingerprint detection means to authorize the actuation of the device only to the authorized person or persons, and thereby prevent any accidental actuation, for example by children. Other recognition means could be envisaged, such as facial recognition.

A cap 25 is provided in order to fix the dispenser 1 in the assembly formed by the inner body 1 and the outer body 20. This cap 25 comprises complementary coupling means, advantageously produced in the form of an internal thread 26 which is adapted to cooperate with the coupling means of the inner body 10.

Thus, in order to assemble the dispenser 1 in the device, the reservoir 2 is inserted inside the hollow cylinder 11 of the inner body 10. The cap 25 is then screwed onto said inner body 10. Advantageously, during this screwing, a radial flange of the dispensing head is clamped between the upper edge of the inner body 10 and the cap 25. This is advantageous by limiting the axial clearance of the dispenser 1 in the device, capable of generating malfunctions. Such assembly is shown in FIGS. 3 and 4.

The device further comprises a push member 30 and an automatic actuating system.

The push member 30 moves axially relative to the inner body 10 between a rest position and an actuating position. A spring 40 is disposed in the inner body 10 between a bottom of said inner body 10 and an upper radial flange 31 of the push member 30. Advantageously, the push member 30 is firstly inserted into the inner body 10 from above, thus being arranged in said inner body 10 directly below the reservoir 2.

The push member 30 includes a blind hollow sleeve 32 disposed around the bottom of the reservoir 2, said hollow sleeve 32 terminating on the top side in said top radial flange 31 and being closed on the bottom side by a bottom wall 33. In the embodiment shown, the bottom wall 33 passes through the bottom opening of the inner body 10. The hollow sleeve 32 is extended axially downwards by an axial rod 34 comprising a set of teeth 35 and a notch 36.

The automatic actuating system comprises a reset gear 50, a pawl 60, a pawl wheel 65, a floating wheel 70, a motor wheel 75, a worm screw 90 and a motor 80.

The reset gear 50 is mounted on the outer body 20 to pivot about a first pivot axis 51, between a reset position and an actuated position. It comprises a first set of teeth 52 interacting with the set of teeth 35 of the axial rod 34 of the push member 30. The reset gear 50 also includes a second set of teeth 53, the function of which is described below.

Advantageously, the cover 60 is mounted on the body 20 to pivot about a pivot axis 61 between an open position and a closed position. The pawl 60 comprises a tooth 62 interacting with the notch 36 of the axial rod 34 of the push member 30. The pawl 60 also includes an oblong opening 63, the function of which is described below.

The pawl wheel 65 is mounted on the outer body 20 to rotate about a first axis of rotation 66, and includes a third set of teeth 67, the function of which is described below. The oblong opening 63 of the pawl 60 is mounted about said first axis of rotation 66, enabling the pawl 60 to move between two end positions relative to the axis of rotation 66 of the pawl wheel 65.

It should be observed that the pawl 60 and the pawl wheel 65 could be made as a single piece. The construction in two separate parts is advantageous in that it makes it possible to reduce the pivoting travel of the pawl 60, and therefore the bulk of the automatic actuating system.

Preferably, the pawl 60 is biased towards its locking position, for example by a resilient element such as a spring or a resilient blade.

The floating wheel 70 includes a second axis of rotation 71 that is movably mounted in an oblong opening 72 of the outer body 20. FIG. 2 illustrates a plate 25 fixed in the outer body 20, and receiving the aforementioned pivot and rotation axes. This plate 25 comprises said oblong opening 72 receiving the second axis of rotation 71 of the floating wheel 70. The floating wheel 70 also includes fourth set of teeth 73 and fifth set of teeth 74. The fourth set of teeth 73 interacts with the third set of teeth 67 of the pawl wheel 65 when the floating wheel 70 rotates in a first direction of rotation, and with the second set of teeth 53 of the reset gear 50 when the floating wheel 70 rotates in the other direction of rotation. The function of the fifth set of teeth 74 is described below.

The motor wheel 75 is mounted on the outer body 20 to rotate about a third axis of rotation 76, which can be seen in FIG. 2. The motor wheel 75 includes a sixth set of teeth 77 interacting with the fifth set of teeth 74 of the floating wheel 70, and a seventh set of teeth 78 the function of which is described below.

A control module (not shown) may be fixed to the outside of said inner body 10 and/or to the inside of said outer body 20. Advantageously, the control module is fixed to the plate 25. This control module actuates the motor 80 connected to the worm screw 90 interacting with said seventh set of teeth 78 of the motor wheel 75.

The motor 80 may be a 3V DC gear motor adapted to cause the worm screw 70 to rotate along two opposite directions. This motor may be powered in any appropriate manner, for example by means of rechargeable or non-rechargeable batteries or accumulators.

The worm screw 90 interacts with the motor wheel 75 to cause it to rotate in the actuating direction at the start of actuation, and in the reset direction at the end of actuation, after dispensing a dose.

In a variant, it is possible to envisage eliminating the worm screw 90 and causing the motor 80 to interact directly with the motor wheel 75.

An electronic board (not shown) could comprise appropriate electronic elements, in particular such as a microprocessor, in order to cause the device to operate, in particular the motor 80 and the screen 91. Advantageously, the electronic board also includes at least one switch (not shown) for detecting the angular position of the worm screw 90. This switch makes it possible to detect the start of actuation. Thus, if, after a predeterminable time following the unlocking of the device, the user does not actuate the device, the latter can automatically return to the locking position. The at least one switch also enables to detect and record the actuation of the device, and this information can be used to lock the device for a predetermined time. Thus, the electronic control module 90 may comprise timing means to allow a fresh actuation only after the expiry of a predeterminable time delay. These timing means may in particular comprise the internal clock of the microprocessor. Optionally, it is possible to add a real time clock component to it. This temporary locking preferably acts on the control of the motor 80, thus preventing the latter from rotating in order to rotate the worm screw 90. In a variation, the control button 85 itself may be deactivated or locked for a predeterminable time. Advantageously, only authorized persons, such as medical personnel, can modify said locking time by having access to the electronic board or via the display. Advantageously, the screen 91 indicates how much time remains before it is possible and/or necessary to take the next dose. Optionally, an audible and/or visual signal may also be provided if the user nevertheless presses the control button in order to attempt to unlock the device.

The operation of the device shown in the drawings will now be described in more detail.

In a normal actuation cycle, the user takes the device in their hand in the rest position, shown in FIG. 3. In this rest position, the spring 40 is compressed and therefore biases the push member 30 axially upwards, but this axial movement is locked by the pawl 60, the tooth 62 of which is arranged in the notch 36 of the push member 30.

To actuate the device, the user must control the control module so as to operate the motor 80. To do this, it presses on a control button, which may advantageously be integrated into a display, or alternatively it may be provided on the outer body 20. This bearing causes the motor 80, and thus the worm screw 90, to rotate in a first actuating direction.

The rotation of the worm screw 90 in the actuating direction causes the motor wheel 75 to rotate in the actuating direction, as shown by the arrow F1 in FIG. 6b.

This in turn causes the floating wheel 70 to rotate in the actuating direction, in the direction of the arrow F2 that can be seen in FIG. 6b. Since the axis of rotation 71 of the floating wheel is mounted to be movable in the oblong opening 72, this rotation in the actuating direction F2 moves the floating wheel 70 in the oblong opening 72 in the direction of arrow F3.

This movement of the floating wheel 70 in the oblong opening 72 disengages the fourth set of teeth 73 of the floating wheel 70 from the second set of teeth 53 of the push gear 50, and engages this fourth set of teeth 73 with the third set of teeth 67 of the pawl wheel 65.

The rotation of the floating wheel 70 in the actuating direction F2 then causes the pawl wheel 65 to rotate in its actuating direction in the direction shown by arrow F4 in FIG. 7b.

This rotation of the pawl wheel 65 in the actuating direction F4 causes the pawl 60 to pivot about its pivot axis 61, with the axis of rotation 66 of the pawl wheel 65 moving in the oblong opening 63 of the pawl 60. This movement of the pawl 60 towards its release position, which can be seen in FIG. 8B, makes it possible to disengage the tooth 62 of the pawl 60 from the notch 36 of the push member 30.

The push member 30 is then automatically moved axially upwards by the spring 40, thereby moving the reservoir 2, and thus actuating the dispensing member 5, as can be seen in FIGS. 9a and 9b. This actuation being performed by the spring 40, it will be performed with the same force and at the same speed on each actuation, which guarantees an identical spray on each actuation.

This upward axial movement of the push member 30 causes the reset gear 50 to rotate in a first direction in the direction of the arrow F6 shown in FIG. 9b. This rotation of the reset gear 50 towards its actuated position is possible thanks to the absence of interaction in this position between the floating wheel 70 and the reset gear 50.

After the dose has been dispensed, the automatic actuating system will be reset for future use.

The motor 80 stops rotating in the actuating direction. Advantageously, it is the axial movement of the push member 30 and/or of the reservoir 2 that triggers a switch suitable for stopping the motor 80.

For resetting, the control module will rotate the motor 80 and therefore the worm screw 90 in a second reset direction, opposite to the first actuating direction.

Rotation of the worm screw 90 in the reset direction causes the motor wheel 75 to rotate in the reset direction opposite to the actuating direction, as shown by arrow F1′ in FIG. 10b.

This in turn causes the floating wheel 70 to rotate in the reset direction in the direction of arrow F2′ as can be seen in FIG. 10b. Since the axis of rotation 71 of the floating wheel is mounted to be movable in the oblong opening 72, this rotation in the actuating direction F2′ displaces the floating wheel 70 in the oblong opening 72 in the direction of arrow F3′.

This movement of the floating wheel 70 in the oblong opening 72 in the direction of the arrow F3′ disengages the fourth set of teeth 73 of the floating wheel 70 from the third set of teeth 67 of the pawl wheel 65, and engages this fourth set of teeth 73 with the second set of teeth 53 of the push gear 50.

Since the pawl wheel 65 and the pawl 60 are no longer meshed in the floating wheel 70, they can return to their starting position.

The rotation of the floating wheel 70 in the reset direction F2′ then causes the push gear 50 to pivot towards its reset position in the direction of arrow F6′ in FIG. 11b.

This rotation of the push gear 50 towards its reset position then moves the push member 30 axially downwards, compressing the spring 40, as shown in FIGS. 11a and 11b.

When the device returns to the rest position, as can be seen in FIGS. 12a and 12b, the tooth 62 of the pawl 60 again locks the push member 30, and the motor 80 stops rotating in the reset direction. Advantageously, it is the axial movement of the push member 30 and/or of the reservoir 2 that triggers a switch suitable for stopping the motor 80.

Advantageously, when the push member 30 returns to the rest position, the motor 80 rotates by a few degrees in the first actuating position, so as to correctly position the finger 62 of the pawl 60 in the notch 36 of the push member 30, so as to guarantee perfect locking.

By virtue of the transmission of the torque from the motor 80 to the push member 30 by means of several gears, the necessary motor torque is reduced, which makes it possible to use a low-power motor.

Preferably, a subsequent actuation will only be possible after the expiry of the locking time which has been predetermined by the electronic control module.

Although the present invention is described above with reference to an advantageous embodiment, naturally various modifications can be applied thereto by the person skilled in the art, without going beyond the scope of the present invention, as defined by the accompanying claims.

1. A device for dispensing a fluid product, comprising:
a fluid product dispenser comprising: a reservoir containing a fluid product; a dispensing head that is provided with a dispensing orifice, said dispensing head being able to move axially relative to said reservoir; and a dispensing member, such as a pump or a valve, that is mounted on said reservoir, said dispensing member being actuated when said reservoir is moved axially upwards relative to said dispensing head;
an inner body comprising a hollow cylinder receiving said dispenser,
an outer body that receives said inner body, a push member, and an automatic actuating system;
said push member being in contact with said reservoir and mounted axially movable relative to said inner body between a rest position and an actuating position, a spring being interposed between said inner body and said push member, said spring being compressed in the rest position of said push member in order to urge it towards its actuating position;
said automatic actuating system comprising a reset gear, a pawl a floating wheel, a motor wheel, and a motor, wherein:
said pawl interacts with said push member to lock it in the rest position, said pawl being moved from its locked position towards a release position by said floating wheel when the motor rotates in a first actuating direction, and
said reset gear interacts with said push member, said reset gear being moved by said floating wheel when the motor rotates in a second reset direction in order to return said push member into its rest position.

2. The device according to claim 1, wherein rotation of said motor in said first actuating direction moves said floating wheel to interact with said pawl, and rotation of said motor in said second reset direction moves said floating wheel to interact with said reset gear.

3. The device according to claim 1, wherein said reset gear is pivotally mounted on said outer body and includes a first set of teeth interacting with a set of teeth of said push member, and a second set of teeth interacting with said floating wheel when said motor rotates in said second reset direction.

4. The device according to claim 1, wherein said pawl is pivotally mounted on said outer body and includes a tooth that interacts with a notch in said push member.

5. The device according to claim 4, wherein a pawl wheel is mounted to rotate on said outer body about an axis of rotation, said pawl including an oblong opening mounted about said axis of rotation, said pawl wheel interacting with said floating wheel when said motor rotates in said first actuating direction.

6. The device according to claim 1, wherein said pawl is biased towards its locking position, in particular by a resilient element such as a spring or an resilient blade.

7. The device according to claim 1, wherein said floating wheel includes a rotation axis movably mounted in an oblong opening of said outer body.

8. The device according to claim 1, wherein said motor wheel is rotatably mounted on said outer body and interacts with said floating wheel when said motor rotates in said first actuating direction or in said second actuating direction.

9. The device according to claim 1, wherein said motor rotates a worm screw which interacts with said motor wheel.

10. The device according to claim 1, comprising a control module to control said motor.

11. The device according to claim 10, wherein said control module includes timing means for preventing, after each actuation of the dispenser, the next actuation of said motor for a predeterminable time.

12. The device according to claim 11, wherein said timing means locks a control button of the device and/or said motor.

13. The device according to claim 1, wherein said spring is arranged in said inner body, between a bottom of said inner body and an upper radial flange of said push member.
